(19) United States
(12) Patent Application Publication
(10) Pub. No.: US 2010/0191272 A1
(43) Pub. Date: Jul. 29, 2010

(54) DISTAL ACCESS EMBOLIC PROTECTION SYSTEM AND METHODS OF USING THE SAME

(75) Inventor: Karl KEATING, Oughterard (IE)

Correspondence Address: Sughrue Mion-ABBOTT LABS, 2100 Pennsylvania Avenue, N.W., Washington, DC, 20037, US

(73) Assignee: SALVIAC LIMITED, Dublin (IE)

(21) Appl. No.: 12/358,279

(22) Filed: Jan. 23, 2009

(51) Int. Cl. A61F 2/01

(52) U.S. Cl. 606/200

(57) ABSTRACT

A distal access embolic protection system and methods of using the same for the capture and removal of embolic material from a blood vessel during an interventional procedure are provided. A distal access embolic protection system is inserted through the wall of the blood vessel at a position distal to a stenosis and a distal access filter device is deployed at the point of insertion. After the filter is deployed and is filtering blood that is flowing across the treatment site, an interventional procedure can be performed at the treatment site. After the procedure, the filter device can be collapsed and removed from the blood vessel. A further method is provided wherein a second embolic protection system including a second filter is advanced to the treatment site using a transcatheter technique after the distal access filter has been deployed. A distal access embolic protection system is also provided.

### FIELD OF THE INVENTION

The present invention relates to medical devices and, more particularly, to a system and methods of using the same for the prevention of embolic events that may be caused by interventional procedures such as angioplasty or stenting.

### BACKGROUND OF THE INVENTION

The term “stroke” is used to describe a medical event whereby blood supply to the brain or specific areas of the brain is restricted or blocked to the extent that the supply is inadequate to provide the required flow of oxygenated blood to maintain function. The brain will be impaired either temporarily or permanently, with the patient experiencing a loss of function such as sight, speech or control of limbs. There are two distinct types of stroke, hemorrhagic and embolic. Embolic stroke may be caused by embolic material that may become dislodged after stenting.

Medical literature describes artery disease as a significant source of embolic material. Typically, an atherosclerotic plaque builds up in the arteries. The nature of the plaque varies considerably, but in a significant number of cases pieces of the plaque can break away and flow distally and, for example, block blood flow to specific areas of the brain and cause neurological impairment. Plaque can also break free and flow into the lungs or heart and cause other adverse events. Treatment of the disease in the carotid artery is classically by way of surgical carotid endarterectomy whereby, the carotid artery is cut and the plaque is physically removed from the vessel. The procedure has broad acceptance with neurological complication rates quoted as being low, somewhere in the order of 5% although claims vary widely on this.

Not all patients are candidates for surgery. A number of reasons may exist such that the patients could not tolerate surgical intervention. In these cases, and in an increasing number of cases where the patients are surgical candidates, the patients are being treated using transcatheter techniques. In this case, the evolving approach uses devices inserted in the femoral artery and manipulated to the site of the stenosis. A balloon angioplasty catheter is inflated to open the artery and an intravascular stent is sometimes deployed at the site of the stenosis. The action of these devices as with surgery can dislodge embolic material which will flow with the arterial blood and if large enough, eventually block a blood vessel and cause a stroke.

In typical carotid percutaneous transluminal angioplasty (PTA) procedures, a guiding catheter or sheath is percutaneously introduced into the cardiovascular system of a patient through the femoral arteries and advanced through the vasculature until the distal end of the guiding catheter is in the common carotid artery. A guidewire and a dilatation catheter having a balloon on the distal end are introduced through the guiding catheter with the guidewire sliding within the dilatation catheter. The guidewire is first advanced out of the guiding catheter into the patient's carotid vasculature and is directed across the arterial lesion. The dilatation catheter is subsequently advanced over the previously advanced guidewire until the dilatation balloon is properly positioned across the arterial lesion. Once in position across the lesion, the expandable balloon is inflated to a predetermined size with a radiopaque liquid at relatively high pressure to radially compress the atherosclerotic plaque of the lesion against the inside of the artery wall and thereby dilate the lumen of the artery. The balloon is then deflated to a small profile so that the dilatation catheter can be withdrawn from the patient's vasculature and the blood flow resumed through the dilated artery. As should be appreciated by those skilled in the art, while the above-described procedure is typical, it is not the only method used in angioplasty.

In angioplasty procedures of the kind referenced above, abrupt reclosure may occur or restenosis of the artery may develop over time, which may require another angioplasty procedure, a surgical bypass operation, or some other method of repairing or strengthening the area. To reduce the likelihood of the occurrence of abrupt reclosure and to strengthen the area, a physician can implant an intravascular prosthesis for maintaining vascular patency, commonly known as a stent, inside the artery across the lesion. Stents are usually delivered in a compressed condition to the target location and then are deployed into an expanded condition to support the vessel and help maintain it in an open position. The stent is usually crimped tightly onto a delivery catheter and transported in its delivery diameter through the patient's vasculature. The stent is expandable upon application of a controlled force, often through the inflation of the balloon portion of the delivery catheter, which expands the compressed stent to a larger diameter to be left in place within the artery at the target location. The stent also may be of the self-expanding type formed from, for example, shape memory metals or super-elastic nickel-titanium (NiTi) alloys, which will automatically expand from a compressed state when the stent is advanced out of the distal end of the delivery catheter into the body lumen.

The above non-surgical interventional procedures, when successful, avoid the necessity for major surgical operations. However, a danger which is always present during these procedures is the potential for particles of the atherosclerotic plaque, which can be extremely friable, breaking away from the arterial wall. For example, during deployment of a stent, the metal struts of the stent can possibly cut into the stenosis and shear off pieces of plaque which become embolic debris that will travel downstream and lodge somewhere in the patient's vascular system. Pieces of plaque material can sometimes dislodge from the stenosis during a balloon angioplasty procedure and become released into the bloodstream. When any of the above-described procedures are performed in the carotid arteries, the release of emboli into the circulatory system can be extremely dangerous and sometimes fatal to the patient. Debris that are carried by the bloodstream to distal vessels of the brain can cause these cerebral vessels to occlude, resulting in a stroke, and in some cases, death. Therefore, although cerebral PTA has been performed in the past, the number of procedures performed has been limited due to the justifiable fear of causing an embolic stroke should embolic debris enter the bloodstream and block vital downstream blood passages.

Medical devices have been developed to attempt to deal with the problem created when debris or fragments enter the circulatory system following treatment utilizing the above-identified procedures. Some techniques which have had success include the placement of a filter or trap downstream from the treatment site to capture embolic debris before it reaches the smaller blood vessels downstream.

For example, it is known to permanently implant a filter in human vasculature, such as the vena cava, to catch embolic material. It is also known to use a removable filter for this purpose. Such removable filters typically comprise umbrella type filters comprising a filter membrane supported on a collapsible frame on a guidewire for movement of the filter membrane between a collapsed position against the guidewire and a laterally extending position occluding a vessel. Examples of such filters are shown in U.S. Pat. No. 4,723,549 to Wholey et al., U.S. Pat. No. 5,053,008 to Bajaj, U.S. Pat. No. 5,108,419 to Reger et al., and U.S. Pat. No. 6,027,520 to Tsugita et al., the disclosures of which are hereby incorporated by reference. Various deployment and/or collapsing arrangements are provided for the umbrella filter.

Improved filter devices such as those shown in U.S. Pat. No. 6,336,934 to Gilson et al., U.S. Pat. No. 6,551,342 to Shen et al. and U.S. Patent Application Publication No. 2003/0065354 to Boyle et al., the disclosures of which are hereby incorporated by reference, have been designed to overcome the shortcomings of the previous filters. For example, in one embodiment, the filter is freely disposed along the length to the guidewire, thereby allowing the guidewire to be moved independently of the filter assembly.

However, advancing delivery catheters through carotid lesions for deployment of these filter devices carries the risk of embolic events. For example, the delivery catheter and/or the guidewire may scrape the lesion and dislodge embolic material. Because the filter has not yet been deployed in the vessel, the dislodged plaque would be free to travel downstream. The possibility of the catheter and/or guidewire scraping the lesion particularly increases when it is necessary to cross a highly built up occlusion in the artery

Another technique developed to deal with the problem created when debris or fragments enter the circulatory system following treatment utilizing the above-identified procedures includes the deployment of a tubular member over the lesion, which protects the lesion by covering the plaque with the tubular member. One such plaque-trapping device is discussed in U.S. Pat. No. 6,592,616 to Stack et al., which is hereby incorporated herein by reference. However, according to the method disclosed by Stack et al., a delivery catheter must cross the lesion, similar to the filter techniques described above. Thus, plaque may be dislodged and form emboli in the bloodstream, particularly when it is necessary to cross a highly built up occlusion in the artery.

In light of the above, it becomes apparent that there remains a need for an improved system and method for the capture and removal of embolic material from a blood vessel during an interventional procedure performed on an area of plaque at a treatment site, which is easy and safe to deploy. Such a device and method would be particularly advantageous if it could be deployed within the vasculature of a patient without the risk of creating embolic material. The inventions disclosed herein satisfy these and other needs.

### BRIEF SUMMARY

The present invention provides a distal access embolic protection system and methods of using the same for the capture and removal of embolic material from a blood vessel created during the performance of a therapeutic interventional procedure, such as a balloon angioplasty or a stenting procedure, to prevent the embolic material from lodging and blocking blood vessels downstream from the interventional site. The present invention is particularly useful while performing an interventional procedure in critical arteries, such as the carotid arteries, with a highly built up stenosis, in which downstream blood vessels can become blocked with embolic debris, including the main blood vessels leading to the brain or other vital organs.

In accordance with an embodiment of the present invention, there is provided a method for the capture and removal of embolic material from a blood vessel during an interventional procedure performed on an area of plaque at a treatment site, comprising:

providing a distal access embolic protection system including an expandable filter slidably disposed within a catheter;

inserting the embolic protection system through a wall of the blood vessel at a position distal to the plaque;

deploying the expandable filter from a collapsed state to an expanded state at the point of insertion by withdrawing the catheter and maintaining the position of the filter to expand the filter to engage a wall of the blood vessel;

performing the interventional procedure at the treatment site after deploying the filter; and

collapsing the filter by advancing the catheter over the filter; and

removing the distal access embolic protection system from the blood vessel.

In accordance with another embodiment of the present invention, there is provided a method where a second embolic protection system including an expandable filter is advanced to the treatment site using a transcatheter technique after the deployment of the distal access embolic protection system described above.

In accordance with a further embodiment of the present invention, there is also provided a distal access embolic protection system including a catheter having a proximal end, a distal end and a lumen extending therebetween, wherein the catheter is adapted for insertion through a wall of a blood vessel; and an expandable filter slidably disposed within the lumen of the catheter, the expandable filter comprising a hoop-shaped expansion frame directly fixed to an operating element and a filter net attached to the frame, wherein the expandable filter is capable of being deployed from a collapsed state within the lumen of the catheter to an expanded state within the blood vessel.

The accompanying figures, which are incorporated in and constitute part of this specification, are included to illustrate and provide a further understanding of the method and system of the invention. Together with the description, the figures serve to explain the principles of the invention.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIGS. 1-3 are cross sectional views of a blood vessel having an area of plaque illustrating the use of a distal access embolic protection system according to one embodiment of the present invention that is inserted into the vessel at a position distal to the area of plaque.

FIGS. 4 and 5 are cross sectional views of a blood vessel having an area of plaque illustrating an embodiment of the present invention employing the combined use of a distal access embolic protection system according to one embodiment of the present invention with a second embolic protection system that is delivered to the treatment site using a conventional transcatheter technique.

FIG. 6 is a cross sectional view of one embodiment of a distal access embolic protection system according to the present invention showing a filter element in a collapsed state within a catheter.

FIG. 7 is a cross sectional view of one embodiment of a distal access embolic protection system according to the present invention showing an expandable filter element in its expanded state.

FIG. 8 is a partial perspective view of one embodiment of a distal access embolic protection system according to the present invention showing an expandable filter element in its expanded state.

FIG. 9 is a perspective view of another embodiment of an expandable filter element in its expanded state.

FIG. 10 is an end view in the direction of arrow A in FIG. 9.

It should be noted that the figures are not drawn to scale and that elements of similar structures or functions are generally represented by like reference numerals for illustrative purposes throughout the figures. It also should be noted that the figures are only intended to facilitate the description of embodiments of the present invention.

### DETAILED DESCRIPTION

It is to be understood that both the foregoing general description and the following detailed description are exemplary and are intended to provide further explanation of the invention claimed.

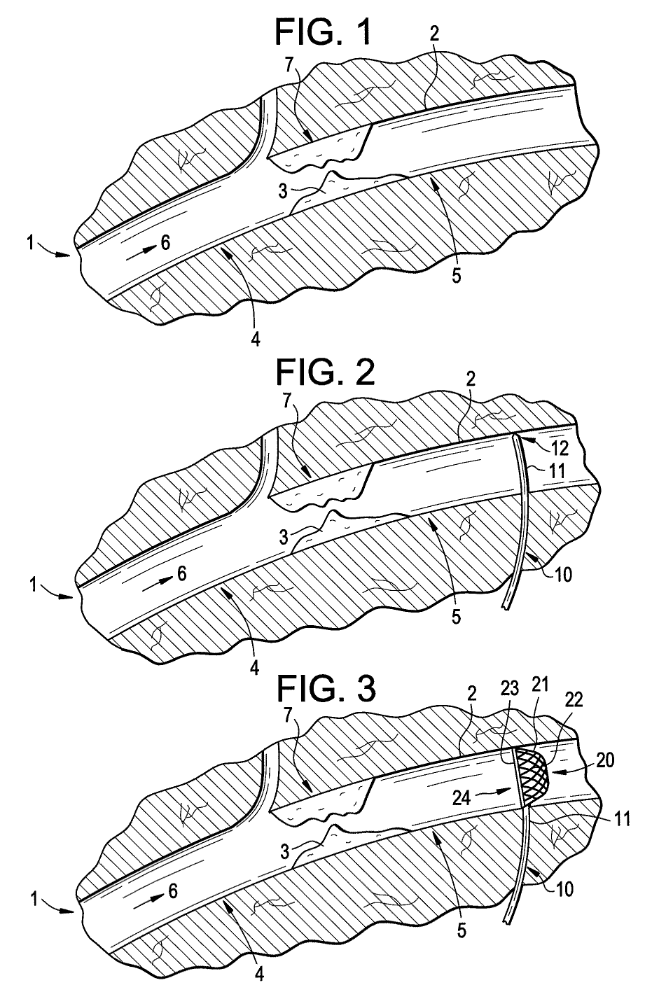

FIG. 1 shows a blood vessel 1 defined by a vessel wall 2. The blood vessel 1 has an area of plaque 3 within the treatment area 7. Blood flows across the plaque 3 in the direction 6, from the proximal side 4 of the plaque 3 to the distal side 5 of the plaque.

Referring to FIGS. 2 and 3, a first embodiment of the present invention is illustrated, namely a distal access embolic protection system 10 including a catheter 11 and a filter device 20 that is inserted through the wall 2 of the blood vessel 1 at a position on the distal side 5 of the plaque 3. By inserting the distal access embolic protection system 10 through the wall 2 of the vessel, it is possible to deliver the medical device to the distal side 5 of the plaque 3 without crossing the treatment site 7 with a catheter, a guidewire or other device. The catheter 11 typically comprises a substantially flexible member having a proximal end (not shown) that extends outside of the body for manipulation by a physician, a distal end 12 within the body and a lumen (not shown) extending therebetween. The catheter 11 to be used with the distal access embolic protection system 10 is not particularly limited, and a suitable structure would be familiar to those skilled in the art of transcatheter filter devices. The filter device 20 is slidably mounted within the lumen of the catheter 11 for delivery in a collapsed state (not shown), as is discussed further below.

Referring to FIG. 3, the filter device 20 is deployed to engage the wall 2 of the vessel at a position on the distal side 5 of the plaque 3 by moving the filter device 20 from the collapsed state within the catheter 11 (as in FIG. 2) to an expanded state (as shown in FIG. 3) for filtering blood flowing through the vessel. The filter device 20 generally comprises an expandable filter 21 on the distal end of the device comprising an expansion frame 23 that supports a filter net 22. When deployed, the expansion frame 23 is expanded across the vessel and defines an opening 27 through which blood and emboli can enter. The blood will pass through the filter net 22, but the openings or pores in the net are sized so as to retain the embolic material. Typically, the expansion frame 23 will be made from a self-expanding material or a shape-memory material, such as Nitinol. In such a case, the filter device 20 can be deployed within the vessel by retracting the catheter 11 in the proximal direction while maintaining the position of the filter device 20 to expose and withdraw the expandable filter 21 at the distal end. Once withdrawn from the catheter 11, the expansion frame 23 is free to expand to engage the wall of the vessel. The distal access embolic protection system 10, and particularly, the filter device 20 are discussed in more detail below.

As illustrated in FIGS. 2 and 3, according to one embodiment of the present invention, a simplified and improved method of directly deploying an embolic filter at a position distal to a lesion is realized. First, the distal access embolic protection system 10 is inserted directly into the blood vessel at a position distal to the lesion. Typically, the embolic protection system 10 enters the blood vessel substantially perpendicular to the wall of the vessel, as shown, and then can be advanced directly across the inside diameter of the vessel to a point on the opposite side of the vessel from the insertion point. Second, the expandable filter 21 can then be expanded within the vessel directly at the point of insertion by withdrawing the catheter 11 while maintaining the position of the expandable filter 21 to expand the filter to engage the wall of the blood vessel. One of ordinary skill would understand that this method is considered a simplified and improved method of deploying an expandable filter at a position distal to a lesion because, essentially, the system is a two step process where (1) the system can be quickly and easily inserted (e.g., pierced or plunged) straight into and advanced across the inside of the vessel and then (2) the catheter can be quickly and easily withdrawn while maintaining the position of the filter to expand and deploy the filter directly at the point of insertion.

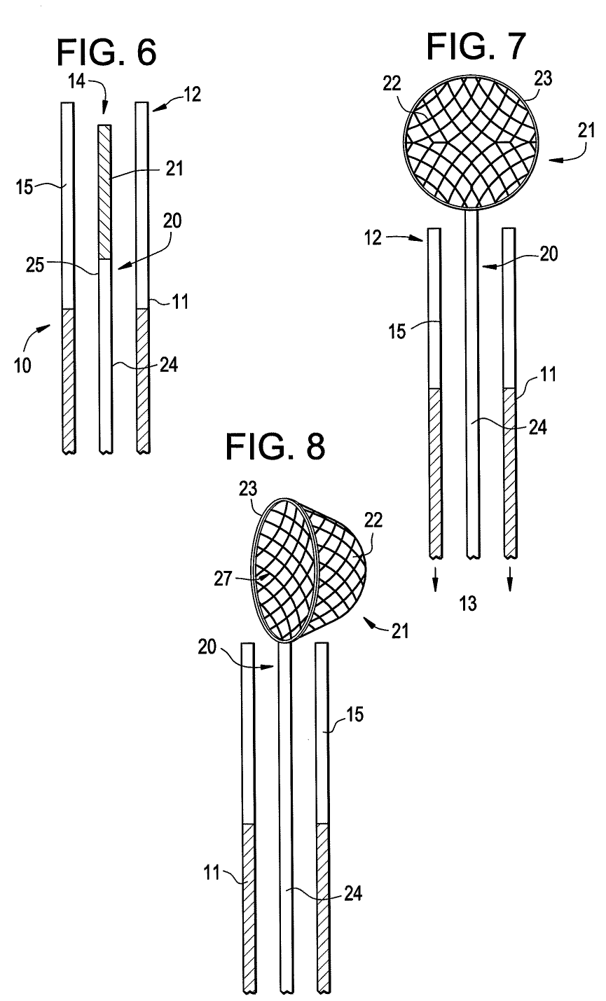

Referring to FIG. 6, there is shown a cross sectional view of one particular embodiment of a distal access embolic protection system 10 according to the invention. The distal access embolic protection system 10 includes a filter device 20 comprising an operating element 24 and an expandable filter 21 at the distal end 25 of the operating element 24. The filter device 20 is slidably disposed within a lumen 14 of a catheter 11, and the expandable filter 21 is shown in a low-profile, collapsed state that is suitable for delivery. The operating element 24 can be an elongated member having a distal end 25 coupled to the expandable filter 21 and a proximal end (not shown) that extends outside of the patient for manipulation by the operating physician. Typically, the expandable filter 21 will be fixedly or rigidly attached to the operating element 24 by any suitable means. A rigid connection between the expansion frame 23 of the expandable filter 21 and the operating element 24 would facilitate the appropriate alignment of the filter within the vessel by mitigating rotation of the device within the vessel. However, the connection between the expandable filter 21 and the operating element 24 is not particularly limited. For example, the expandable filter 21 and the operating element 24 may not be separate elements, or in the alternative, the expandable filter 21 may be flexibly or rotatably coupled to the operating element 24. The expandable filter 21 is discussed further below.

As shown in FIGS. 6-9, the distal end 12 of the catheter 11 may comprise an expandable tip portion 15, as described by U.S. Pat. No. 7,094,243 to Mullholland et al., the disclosure of which is hereby incorporated herein by reference. The expandable tip portion 15 allows the catheter 11 to retrieve or deliver a device that might be larger than the catheter 11 itself.

Referring to FIG. 7, the filter device 20 is slidably disposed within the lumen 14 of the catheter 11 as shown in FIG. 6, but the catheter 11 has been retracted in the proximal direction 13 to uncover and withdraw the expandable filter 21. The expandable filter 21 generally comprises an expansion frame 23 and a filter net 22 connected to the frame. The expandable filter 21 shown in FIG. 7 is a “hoop filter” having the shape of a hoop (i.e., a round, substantially circular shape or an elliptical shape) defined by the expansion frame 23. As shown in FIGS. 2-5, having the hoop-shaped expansion frame 23 directly fixed to the end of the operating element 24 allows the expandable filter 21 to be directly inserted (as shown, substantially perpendicularly inserted) through the vessel wall and into the vessel for immediate deployment at the insertion site. By directly fixed it is meant that, if the expansion frame 23 has a circular shape, the operating element 24 would be directly connected to a portion of the circumference of the circular expansion frame 24, as shown in FIGS. 7 and 8. Thus, when the expansion frame 23 is directly fixed to the operating element 24, the opening 27 would be essentially parallel to the operating element 24 (in contrast, with a conventional umbrella filter, the opening of the filter would be perpendicular to its operating element, such as a guidewire).

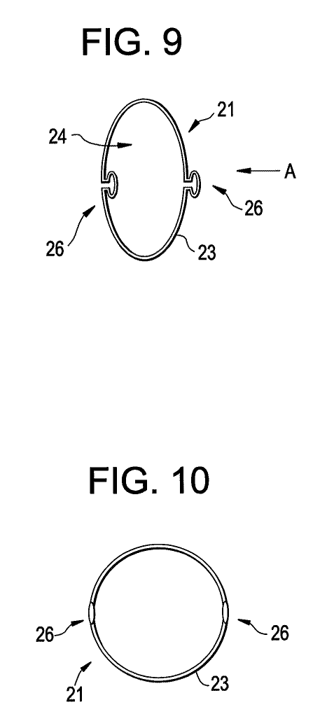

Another embodiment of the expandable filter 21 is illustrated in FIGS. 9 and 10. The hoop-shaped expansion frame 23 may have one or more strain distributing linkage elements, as described by U.S. Pat. No. 6,964,672 to Brady et al., the disclosure of which is hereby incorporated herein by reference. In FIGS. 9 and 10, the linkage element comprises a loop 26. The loop 26 in this case extends axially and distally of the hoop-shaped expansion frame 23. The loop 26 is of generally omega shape as illustrated. The loop 26 can function as a strain reliever or distributor when the expansion frame 23 is collapsed as illustrated in FIG. 6. The loop 26 has a relatively large radius resulting in highly efficient strain distribution. In addition, the loop 26 allows the support frame to accommodate varying vessel contours and sizes. In effect the loop 26 acts as a diameter or circumference adjuster allowing an embolic protection device to adapt to difference vessel contours and sizes while maintaining a position with the vessel wall. The strain relieving geometry of the loops 26 enhances the compliance of the bend points without creating a weakened hinge point, thus ensuring that there is no discontinuity in the circumferential seal against the vessel wall.

However, the shape of the expandable filter 21 is not particularly limited, such that the expansion frame 23 is adapted to engage the vessel wall upon expansion. In this regard, the expansion frame 23 may be made from any suitable material, but is typically made from a self-expanding material or a shape-memory material, such as Nitinol.

The filter net 22 shown in FIGS. 7 and 8 is not particularly limited so long as it includes openings or pores that are sized to retain embolic material but allow blood to pass freely through net. As should be appreciated by those skilled in the art, appropriate materials for the distal access embolic protection device 10 and, more particularly, the filter device 20 comprising the expansion frame 23 and the filter net 22, may be chosen from any of the references teaching removable filters that were discussed above and have been incorporated by reference. For example, the filter net 22 may be of an oriented polymeric material, as described in WO 01/97714A and U.S. Patent Application Publication No. 2002/0042627A to Brady et al., the disclosures of which are hereby incorporated herein by reference.

Referring to FIG. 8, there is shown a partial perspective view of one embodiment of the distal access embolic protection system 10 according to the present invention. While the size and shape of the filter net 22 is not particularly limited, as seen in FIG. 8, the filter net 22 attached to the expansion frame 23 of the filter device 20 may have a bag-like shape (i.e., a flexible container with a single opening, defined by the expansion frame, that is suitable for trapping embolic material). Also illustrated in FIG. 8 is the opening 27 through which blood and emboli can enter. When the catheter 11 is retracted to expose the expandable filter 21, the expansion frame 23 expands to engage the wall of the vessel and blood will enter the opening 27 and then pass through the filter net 22, but the openings or pores in the filter net 22 are sized so as to retain the embolic material.

To retrieve the filter device 21 from within the vessel when the distal access embolic protection system 10 is no longer required, the catheter 11 can be advanced in the distal direction (not shown) to collapse the expansion frame 23 and draw the filter device 21 back into the lumen 14 of the catheter 11 (e.g., the low-profile, collapsed state is shown in FIG. 6). With embolic material safely within the filter net 22 that is collapsed within the catheter 11, the distal access embolic protection system 10 may be withdrawn from the patient by the physician. Further, having a hoop-shaped expansion frame 23 directly coupled to the operating element 24 of the filter device 21 simplifies the collapsing and retrieving filter.

Once the distal access embolic protection system 10 is deployed in the vessel and blood is being filtered through the filter device 20, an interventional procedure can be performed at the treatment site 7, such as angioplasty or stenting, by any known technique. For example, a variety of suitable expandable or self-expanding stents as are conventionally known can be used with the invention, and details can be found in U.S. Pat. No. 6,375,676 to Cox, incorporated herein by reference in its entirety. When a stent is delivered to the treatment site by a transcatheter technique, for example, emboli that are dislodged when the catheter, guidewire, etc. crosses the plaque can be safely trapped by filter device 20 of the distal access embolic protection device 10.

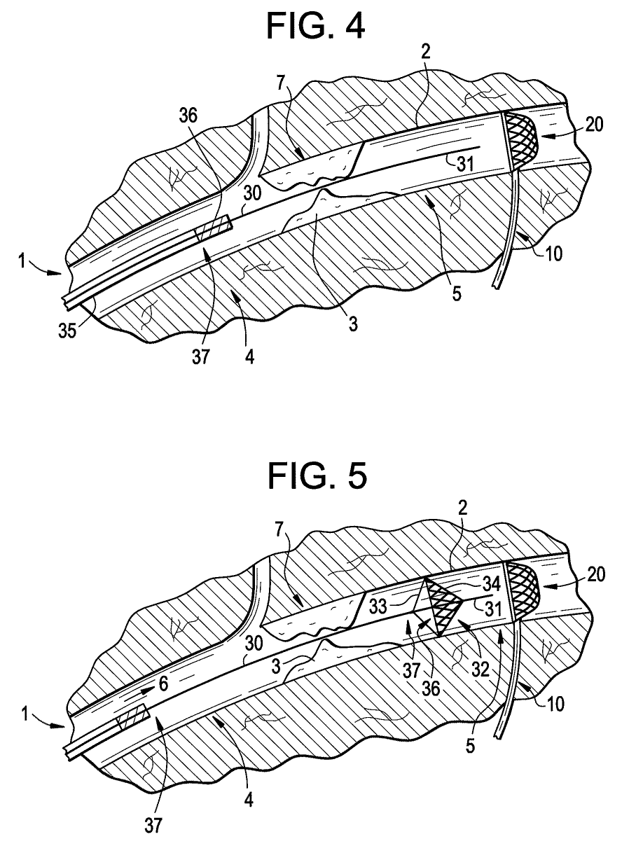

Alternatively, as shown in FIGS. 4 and 5, before one or more interventional procedures are performed at the treatment site 7, a second embolic protection system 37 may be advanced to the treatment site 7 using a conventional transcatheter technique. In such a case, the expandable filter 21 of the distal access embolic protection system 10 might be considered a “preliminary filter” or a “temporary filter” that functions to trap embolic material that may become dislodged during placement of the second embolic protection system 37, which could be considered a “main filter” or a “primary filter.”

For example, in reference to FIGS. 4-5, there is illustrated an embodiment of the present invention wherein a second embolic protection system 37 including a second filter device 32 is advanced to the treatment site 7 over a guidewire 30. A variety of suitable filters can be used as the second filter device 32 of the invention, and details can be found, for example, in any one of U.S. Pat. Nos. 4,723,549, 5,053,008, 5,108,419, 6,027,520, 6,336,934 and 6,551,342 or U.S. Patent Application Publication No. 2003/0065354, discussed above. Typically, the second filter device 32 is disposed over a guidewire 30 and is delivered to the treatment site 7 in a collapsed condition (not shown) within the distal end 36 of a delivery catheter 35. The delivery catheter 35 can be advanced to the treatment site 7 by translation along the guidewire 30 by any suitable means known in the art, such as through the use of a rapid exchange catheter (not shown). The delivery catheter 35 crosses the plaque 3 and can be expanded within the vessel to filter blood by any known method. For example, the second filter device 32 can be made from a self-expanding material capable of expanding to engage a wall of the vessel 2. Accordingly, the second filter device 32 may be deployed within the vessel by withdrawal from the distal end 36 of the delivery catheter 35 by retracting the catheter 35 in the distal direction while maintaining the position of the second filter device 32. After deploying the second filter device 32, the delivery catheter 35 can be withdrawn from the patient so that another medical device(s) may be translated along the guidewire 30 to the treatment site 7.

As shown in FIG. 5, the second filter device 32 generally comprises an expansion frame 33 and a flexible filter net 34 attached to the frame 33. The second filter device 32 includes an opening(s) 36 for entry of blood and emboli, and the filter net 34 is sized to permit the flow of blood but trap the emboli. However, the structure of the second filter device 32 is not particularly limited.

According to this embodiment of the invention, before crossing the plaque 3 with the second filter device 32, the physician would first deploy the distal access filter device 20 discussed above. Next, the second filter device 32 can cross the plaque 3 and be deployed from the collapsed state (not shown) to the expanded state (as shown) at a position within the vessel that is distal to the plaque 3 but is proximal to the distal access filter device 20.

After the interventional procedure(s) is completed and/or the use of the second filter device 32 is no longer required, a retrieval catheter (not shown), which may be the same as the delivery catheter, is advanced along the guidewire 30 and slid over the second filter device 32 to close the openings 36 and then gradually collapse the filter net 34 as the retrieval catheter advances over the second filter device 32, as is well known to those skilled in the art. Once the second filter device 32 is fully loaded in the retrieval catheter, it can then be withdrawn from the patient along with any embolic material safely trapped within the second filter device 32.

As should be apparent to those skilled in the art, the distal access embolic protection system 10 may be withdrawn any time after the interventional procedure(s) is performed or any time after the second filter device 32 is deployed in the vessel. For example, the distal access embolic protection system 10 could be used simply as a “preliminary filter” to trap emboli that may be dislodged during the deployment of the second filter device 32. In such a case, the distal access embolic protection system 10 could be removed after the second filter device 32 is deployed to filter blood flowing across the treatment site. Alternatively, the distal access embolic protection device 10 could remain in the vessel until the second filter device 32 is removed. In such a case, any embolic material that might pass through or around the second filter device 32 during the interventional procedure(s) and/or become dislodged upon withdrawal of the second embolic protection system 37 from the patient would be safely trapped by the distal access embolic protection device 10. For example, the distal access emboli protection device 10 can trap plaque that might extrude between the struts into the open areas of a stent and become dislodged upon retrieval of the second filter device 32, as is described in U.S. patent application Ser. No. 12/211,253, herein incorporated by reference in its entirety.

In addition, one of ordinary skill in the art would understand that in order to achieve some of the objects of the embodiment of the invention illustrated in FIGS. 4 and 5, it would be possible to employ any appropriate filter as either the distal access filter and the secondary filter.

In conventional transcatheter techniques, wherein devices are typically inserted in the femoral artery and manipulated to the site of the stenosis, upon removal of the device from the body, a vessel closure suture is typically deployed. Likewise, upon removal of the distal access embolic protection system 10 according to the invention a vessel closure suture would be typically deployed. One such example of a method for the percutaneous suturing of a vascular puncture site is disclosed in U.S. Pat. No. 5,417,699 to Klein et al., which is incorporated herein by reference and which describes embodiments of a Perclose®-type vessel closure device. In one embodiment, a vessel closure device is actuated on removal of the distal access embolic protection system 10.

In accordance with the above description, a distal access embolic protection system and methods of using the same for the safe and effective capture and removal of embolic material from a blood vessel during an interventional procedure performed on an area of plaque at a treatment site have been realized.

The invention is susceptible to various modifications and alternative means, and specific examples thereof have been shown by way of example in the drawings and are herein described in detail. It should be understood, however, that the invention is not to be limited to the particular devices or methods disclosed, but to the contrary, the invention is to cover all modifications, equivalents, and alternatives falling within the spirit and scope of the claims.

What is claimed is:

1. A method for the capture and removal of embolic material from a blood vessel during an interventional procedure performed on an area of plaque at a treatment site, comprising:
providing a distal access embolic protection system including an expandable filter slidably disposed within a catheter;
inserting the distal access embolic protection system through a wall of the blood vessel at a position distal to the plaque;
deploying the filter from a collapsed state to an expanded state at the point of insertion by withdrawing the catheter and maintaining the position of the filter to expand the filter to engage a wall of the blood vessel;
performing the interventional procedure at the treatment site after deploying the filter; and
collapsing the filter by advancing the catheter over the filter; and
removing the distal access embolic protection system from the blood vessel.

2. The method according to claim 1, wherein the interventional procedure includes deploying an implantable stent at the treatment site.

3. The method according to claim 1, wherein the distal end of the catheter comprises an expandable tip portion.

4. The method according to claim 3, wherein the filter is self expanding.

5. The method according to claim 1, wherein the filter includes a filter net attached to the hoop-shaped expansion frame.

6. The method according to claim 1, further comprising:
closing the blood vessel upon removal of the distal access embolic protection system.

7. The method according to claim 1, wherein the embolic protection system enters the blood vessel substantially perpendicular to the wall of the vessel and is advanced directly across the inside diameter of the vessel to a point on the opposite side of the vessel from the insertion point.

8. A method for the capture and removal of embolic material from a blood vessel during an interventional procedure performed on an area of plaque at a treatment site, comprising:
providing a distal access embolic protection system including a distal access filter;
inserting the distal access embolic protection system through a wall of the blood vessel at a position distal to the plaque;
deploying the distal access filter from a collapsed state to an expanded state at the position distal to the plaque;
providing a second embolic protection system including a second filter;
advancing the second embolic protection system to a position proximal to the plaque using a transcatheter technique;
crossing the plaque with the second embolic protection system after the distal access filter is deployed;
deploying the second filter from a collapsed state to an expanded state at a position distal to the plaque and proximal to the distal access filter;
performing an interventional procedure at the treatment site after the second filter is deployed in the blood vessel;
collapsing the distal access filter and removing the distal access embolic protection system from the blood vessel; and
collapsing the second filter and removing the second embolic protection system from the blood vessel.

9. The method according to claim 8, wherein the interventional procedure includes deploying an implantable stent at the treatment site.

10. The method according to claim 8, wherein the distal access embolic protection system is removed from the blood vessel after the second filter is deployed.

11. The method according to claim 8, wherein the distal access embolic protection system is removed from the blood vessel after the interventional procedure is performed.

12. The method according to claim 8, wherein the distal access embolic protection system is removed from the blood vessel after the second embolic protection system is removed from the blood vessel.

13. The method according to claim 8, wherein the second filter is disposed on a guidewire and the guidewire can be moved independently of the second filter.

14. The method according to claim 8, wherein the distal access filter comprises an expansion frame and a filter net attached to the frame.

15. The method according to claim 14, wherein the expansion frame is made from a self-expanding material.

16. The method according to claim 14, wherein the expansion frame is hoop-shaped.

17. The method according to claim 8, further comprising:
closing the blood vessel upon removal of the distal access embolic protection system.

18. A distal access embolic protection system comprising:
a catheter having a proximal end, a distal end and a lumen extending therebetween, wherein the catheter is adapted for insertion through a wall of a blood vessel; and
an expandable filter slidably disposed within the lumen of the catheter, the expandable filter comprising a hoop-shaped expansion frame directly fixed to an operating element and a filter net attached to the frame, wherein the expandable filter is capable of being deployed from a collapsed state within the lumen of the catheter to an expanded state within the blood vessel.

19. The distal access embolic protection system according to claim 18, wherein the expandable filter is self expanding.

20. The distal access embolic protection system according to claim 18, wherein the distal end of the catheter comprises an expandable tip portion.
